(19) United States
(12) Patent Application Publication
(10) Pub. No.: US 2024/0228696 A1
(43) Pub. Date: Jul. 11, 2024

(54) METHODS FOR THE PRODUCTION OF SUCCINIC ACID FROM SUGARS

(71) Applicant: Solugen, Inc., Houston, TX (US)

(72) Inventors: Kevin Loftis, Spring, TX (US); Sean Hunt, Houston, TX (US); Parth Patel, Seabrook, TX (US); Peter Nguyen, Houston, TX (US); Hans-Joerg Woelk, Houston, TX (US)

(73) Assignee: Solugen, Inc., Houston, TX (US)

(21) Appl. No.: 18/288,839

(22) PCT Filed: Apr. 28, 2022
(86) PCT No.: PCT/US2022/026798

§ 371 Date: Oct. 30, 2023

Related U.S. Application Data
(60) Provisional application No. 63/180,996, filed on Apr. 28, 2021.

(51) Int. Cl. C08G 63/16; C07C 51/31; C07C 51/373; C07D 307/48

(52) U.S. Cl. CPC C08G 63/16; C07C 51/31; C07C 51/373; C07D 307/48

(57) ABSTRACT

A method of preparing succinic acid; the method comprising contacting glucose with an oxidizing catalyst under conditions to produce a first oxidized product; converting the first oxidized product to an aldehyde; and contacting the aldehyde with an acid catalyst under conditions suitable for the formation of succinic acid.

### CROSS-REFERENCE TO RELATED APPLICATIONS

This application is a 35 U.S.C. § 371 national stage application of PCT/US2022/026798 filed Apr. 28, 2022, and entitled “METHODS FOR THE PRODUCTION OF SUCCINIC ACID FROM SUGARS,” which claims priority to U.S. Provisional Application Ser. No. 63/180,996 filed Apr. 28, 2021 and entitled “METHODS FOR PRODUCTION OF SUCCINIC ACID FROM DEXTROSE,” each of which is hereby incorporated herein by reference in its entirety for all purposes.

### TECHNICAL FIELD

The present disclosure relates generally to compositions and methods for producing succinic acid. More specifically, the present disclosure relates to compositions for the production of high purity succinic acid.

### BACKGROUND

Succinic acid is a dicarboxylic acid with the chemical formula (CH₂)₂(CO₂H)₂. In living organisms, succinic acid takes the form of an anion, succinate, which has multiple biological roles as a metabolic intermediate being converted into fumarate by the enzyme succinate dehydrogenase in complex 2 of the electron transport chain. The electron transport chain is involved in making ATP, and as a signaling molecule reflecting the cellular metabolic state. Succinic acid also has uses in antifreeze products, solvents, metallurgy, and in animal feeds. Succinic acid can be used in both deicing, as well as in antifreeze compositions decreasing the usage of toxic ingredients. Solvents that derive from succinic acid include dimethyl succinate, diethylsuccinoylsuccinate, succinimide, pyrrolidine, tetrahydrofuran (THF), 1,4-Butanediol (1-4 BDO), succinonitrile, and γ-butyrolactone (GBL). In metallurgy, succinic acid is used to improve froth floating of ores. In animal feeds, succinic acid outperformed other organic acids as a replacement to antibiotics in feeds.

Fossil fuel-based succinic acid has been produced by oxidation of n-butane into maleic anhydride and maleic acid followed by hydrogenation using H₂ gas. Biologically-based succinic acid has more potential for food applications. These biologically-based production methods use fermentation of commodity sugars (dextrose and fructose base). This means for each mol of succinic acid produced, 2 mols of CO₂ will be produced in a best-case scenario. The biologically-based methods are economically unfeasible due scalability issues associated with fermentation. Thus, an ongoing need exists for methods and compositions for the production of high-purity succinic acid.

### BRIEF DESCRIPTION OF DRAWINGS

For a detailed description of the aspects of the disclosed processes and systems, reference will now be made to the accompanying drawings in which:

FIG. 1 is a schematic illustration of chemical reactions of the present disclosure.

FIG. 2 is a schematic illustration of a process flow diagram for carrying out aspects of the methods disclosed herein.

FIG. 3 graphically depicts the weight percent of dehydrated fructose as a function of time for the samples from Example 1.

FIG. 4 is a plot of the percentage conversion of sodium gluconate to 2-keto gluconate.

### SUMMARY

A method of preparing succinic acid may comprise contacting glucose with an oxidizing catalyst under conditions to produce a first oxidized product; converting the first oxidized product to an aldehyde; and contacting the aldehyde with an acid catalyst under conditions suitable for the formation of succinic acid.

### DETAILED DESCRIPTION

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997) can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Groups of elements of the periodic table are indicated using the numbering scheme indicated in the version of the periodic table of elements published in Chemical and Engineering News, 63(5), 27, 1985. In some instances, a group of elements can be indicated using a common name assigned to the group; for example alkali metals for Group 1 elements, alkaline earth metals for Group 2 elements, transition metals for Group 3-12 elements, and halogens for Group 17 elements, among others.

Regarding claim transitional terms or phrases, the transitional term “comprising,” which is synonymous with “including,” “containing,” “having,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention. A “consisting essentially of” claim occupies a middle ground between closed claims that are written in a “consisting of” format and fully open claims that are drafted in a “comprising” format. Absent an indication to the contrary, when describing a compound or composition “consisting essentially of” is not to be construed as “comprising,” but is intended to describe the recited component that includes materials which do not significantly alter the composition or method to which the term is applied. While compositions and methods are described in terms of “comprising” various components or steps, the compositions and methods can also “consist essentially of” or “consist of” the various components or steps.

In the specification and claims, the terms “a,” “an,” and “the” are intended, unless specifically indicated otherwise, to include plural alternatives, e.g., at least one, or one or more. For any particular compound disclosed herein, the general structure or name presented is also intended to encompass all structural isomers, conformational isomers, and stereoisomers that can arise from a particular set of substituents, unless indicated otherwise. Additionally, the reference to a general structure or name encompasses all enantiomers, diastereomers, and other optical isomers whether in enantiomeric or racemic forms, as well as mixtures of stereoisomers, as the context permits or requires. For any particular formula or name that is presented, any general formula or name presented also encompasses all conformational isomers, regioisomers, and stereoisomers that can arise from a particular set of substituents.

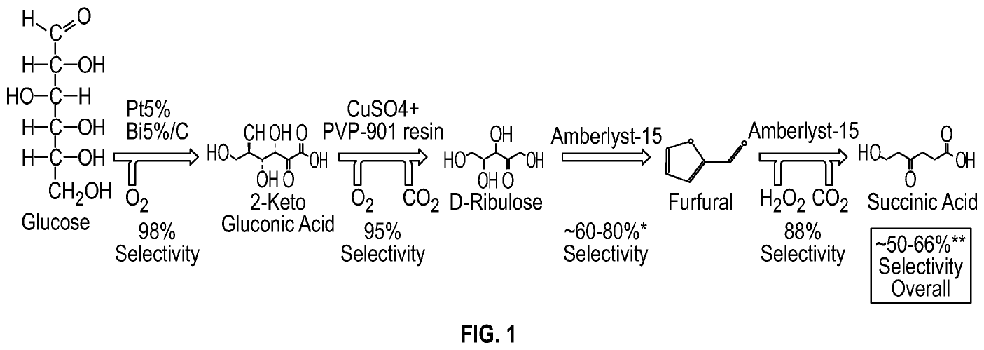

Disclosed herein are methods and compositions for the production of succinic acid. In an aspect, methods of the present disclosure comprise the conversion of sugars (e.g., glucose) to intermediates that are subsequently converted to succinic acid. An overview of the presently disclosed process is presented in FIG. 1. The current compositions and methodologies disclosed result in a commodity chemical derived from renewable resources being produced with greater selectivity and reduced environmental effects when compared to other methods of production of the chemical.

In an aspect, with reference to FIG. 1, a method of the present disclosure comprises oxidation of glucose to form a 2-keto intermediate, 2-keto gluconic acid. In an aspect, the oxidation of glucose is catalyzed by a transition metal catalyst (TMC). Any suitable TMC capable of oxidizing dextrose to produce a 2-keto intermediate may be employed. In an aspect, the TMC comprises a platinum-catalyst, bismuth-catalyst, gold-catalyst, a catalyst having a combination of platinum, bismuth, and/or gold, or a combination thereof. In such aspects, the TMC may be supported on materials such as carbon or alumina. In an aspect, the TMC is a carbon-supported Pt/Bi/Au catalyst. In some aspects, the glucose may be contacted with a supported-Pt/Bi/Au catalyst, or other suitable catalyst, under conditions suitable to yield the formation of 2-keto-gluconic acid.

In an additional or alternative aspect, the TMC catalyst is replaced by an enzyme catalyst, alternatively an alcohol oxidase (AOX, E.C. 1.1.3.13) or alcohol oxidase homolog. AOX is a ubiquitous flavin-dependent enzyme that oxidizes lower primary alcohols to aldehydes using oxygen as an oxidizing agent. AOX may be sourced from methylotrophic yeast of the species Kloeckera, Torulopsis, Candida, Pichia, Hanseniaspora, and Metschnikowia. In an alternative aspect, the AOX is sourced from methanol-utilizing bacteria such as Methylococcus capsulatus, thermophilic soil fungi such as Thermoascus aurantiacus, and brown rot fungus such as Gloeophyllum trabeum. Alternatively, the AOX may be sourced from the white-rot basidiomycete Phanerochaete chrysosporium. Contacting of dextrose with an AOX under suitable conditions results in the formation of the 2-keto intermediate, 2-keto-gluconate.

In some aspects, conversion of dextrose to the 2-keto intermediate (i.e., 2-keto gluconic acid, 2-keto gluconate) occurs with a selectivity to the 2-keto intermediate of equal to or greater than about 80%, alternatively equal to or greater than about 90%, or alternatively from about 80% to about 98%.

In an aspect, the methods of the present disclosure further comprise decarboxylation of the 2-keto intermediate to form D-ribulose. In an aspect, decarboxylation of the 2-keto intermediate is carried out in the presence of any suitable catalyst. For example, the decarboxylation may be carried out in the presence of copper ions (e.g., CuSO₄) in association with a polymer matrix (e.g., PVP-901 resin which is a polyvinylpyrrilidone based resin). In an aspect, the decarboxylation of 2-ketogluconic acid is carried in the presence of oxygen and carbon dioxide and catalyzed by a CuSO₄/PVP-901 resin under conditions suitable for the production of D-ribulose. The reaction may occur with a selectivity for D-ribulose of from about 35% to about 95%, alternatively equal to or greater than about 80%, alternatively equal to or greater than about 90%, or alternatively from about 80% to about 95%.

In an aspect, the methods of the present disclosure further comprise dehydration of D-ribulose to generate furfural. Dehydration of D-ribulose may be carried out under suitable conditions in the presence of an acid catalyst. An acid catalyst suitable for use in the present disclosure is a solid acid catalyst, alternatively an ion-exchange resin acid catalyst. For example, the acid catalyst may comprise an AMBERLYST™ 15DRY Polymeric Catalyst, which is a bead-form, strongly acidic catalyst. In an alternative aspect, the dehydration of D-ribulose is carried out in the presence of formic acid and an AMBERLYST™ 15DRY Polymeric Catalyst. In such aspects, the formic acid may be removed with the addition of an oxidant such as hydrogen peroxide. Conversion of D-ribulose to furfural using the presently disclosed methods and compositions may result in a selectivity to production of furfural of equal to or greater than about 50%, alternatively equal to or greater than about 60%, or alternatively from about 60% to about 80%.

In an aspect, the methods of the present disclosure further comprise oxidation of furfural to generate succinic acid. In an aspect, oxidation of furfural to succinic acid is carried out in the presence of an oxidant, such as hydrogen peroxide, and a catalyst. For example, the reaction mixture may comprise furfural, an acid catalyst comprising AMBERLYST™ 15DRY Polymeric Catalyst, hydrogen peroxide and carbon dioxide. The reaction may be carried out under conditions suitable for the conversion of furfural to succinic acid. Conversion of furfural to succinic acid using the presently disclosed methods and compositions may have a selectivity for formation of succinic acid of equal to or greater than about 50%, alternatively greater than about 60%, or alternatively from about 60% to about 90%.

In an aspect, the selectivity overall for the production of succinic acid from glucose is equal to or greater than about 40%, alternatively equal to or greater than about 45%, or alternatively from about 50% to about 60%.

In some aspects, the presently disclosed compositions and methods allow for the production of succinic acid with greater selectivity when compared to the traditional fermentation processes for the production of succinic acid. Further, the processes disclosed herein are characterized by a significantly lower carbon footprint when compared to the traditional fermentation processes for the production of succinic acid. Additionally, the methods disclosed herein for production of succinic acid are economically advantageous when compared to the traditional fermentation processes for the production of succinic acid. Specifically, the processes disclosed herein result in highly purified materials which can be easily processed to provide high purity succinic while conventional methods using fermentation provide mixtures that must be subjected to costly processing to form a high purity product.

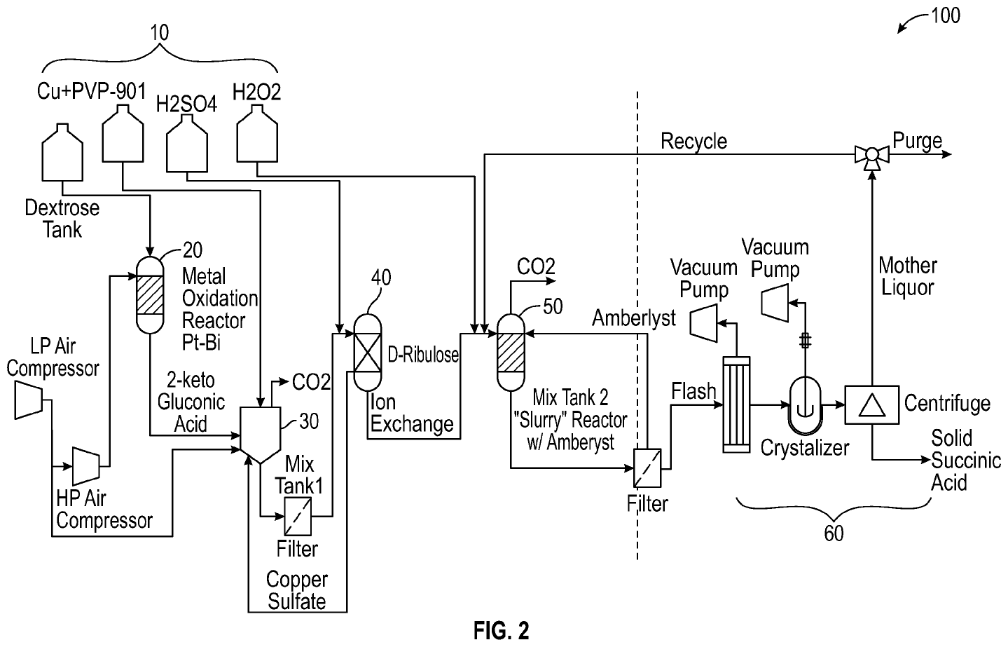

A further advantage of the processes disclosed herein is that the conversion of glucose to succinic acid can occur in a one-pot synthesis. The result is a reduced process complexity. FIG. 2 is a schematic illustration of an aspect of a process flow diagram (PFD) for carrying out the presently disclosed methods (100). With reference to FIG. 2, a series of reactants (10) is introduced to an metal oxidation reactor, 20, where 2-ketogluconic acid is formed and conveyed to a mix tank, 30, where other reactants are introduced to form a reactive mixture which is then conveyed to an ion exchange resin, 40, where D-ribulose is formed and conveyed to a second mix tank before additional processing for form fufural and succinic acid, 60. Each component of the process flow diagram is labeled such that tanks that allow for the introduction of reactants or collection of products are labeled with the reactant name while reactors are also labeled. Flow lines in the diagram indicate the various input and output points for the indicated equipment. It should be appreciated that at the PFD level of detail, not all process interconnections are shown such as spillbacks, block and bleeds, recycle lines, control valves, cooling/heating elements, pumps, intermediate tankage, antifoam, etc.

As one of the United States Department of Energy platform chemicals, succinic acid has various direct and intermediate uses. Herein a platform chemical is defined as a chemical that can serve as a substrate for the production of various other higher value-added products. In one or more aspects, succinic acid may be used as a reactant in the production of polymeric materials. An example of monomers that can be produced from succinic acid include without limitation 2,5-dihydroxyterephthalic acid and 2,5-dimethoxyterephthalic acid.

Additionally or alternatively, example of a polymeric material that can be produced from succinic acid is polybutylene succinate (PBS). PBS is a biodegradable polyester with comparable properties to those of polypropylene. PBS is a “green” plastic that is produced by esterifying succinic acid with 1,4-butanediol (1,4 BDO). In one or more aspects, the reactor systems disclosed herein may be adjusted for the production of PBS by the introduction of a hydrogenation reactor or polycondensation reactor.

In one or more aspects, succinic acid as produced herein may be utilized within and/or to produce a variety of end-use products such as antifreeze products, solvents, metallurgy, and animal feeds. In some aspects, succinic acid prepared as disclosed herein is used in the production of solvents such as and without limitation dimethyl succinate, diethylsuccinoylsuccinate, succinimide, pyrrolidine, tetrahydrofuran, succinonitrile, and gamma-butyrolactone. In an aspect, the methodologies disclosed herein result in succinic acid having a purity of equal to or greater than about 80%, alternatively equal to or greater than about 90%, or alternatively form about 80 to about 95%.

Additionally or alternatively, in an aspect, the glucose feedstock may include copper ions which may be subsequently chelated by the 2-keto gluconic acid produced during the reaction. Referring to FIG. 2, the last reactor, 60, may contain a vinylpyridine resin resulting in the downstream mixing and chelation of the metal ions (e.g., copper ions) by the exchange resin. The recaptured metal ions may be recycled thereby negating the need for a second tank, 40, and improving the continuity of the process.

Additionally or alternatively, in an aspect, dehydration of D-ribulose to produce furfural may be carried out in a solvent such as dimethyl sulfoxide, formic acid, methyl isobutyl ketone, tetrahydrofuran, sulfolane, and 2-butanol. In such aspects, a portion of the water may be removed during heating in the decarboxylation step to improve selectivity.

In some aspects, enzymatic catalysts may be substituted for at least some portion of the transition metal catalysts disclosed herein. For example, the transition metal catalyst may be replaced with a glucose oxidase for the production of 2-keto gluconate or a xylonate dehydratase on the pentose/ketose may replace the transition metal catalyst in the production of furfural.

### EXAMPLES

The subject matter having been generally described, the following examples are given as particular aspects of the disclosure and are included to demonstrate the practice and advantages thereof, as well as aspects and features of the presently disclosed subject matter. It should be appreciated by those of skill in the art that the techniques disclosed in the examples which follow represent techniques discovered by the inventors to function well in the practice of the present subject matter, and thus can be considered to constitute preferred modes for its practice. However, those of skill in the art should, in light of the present disclosure, appreciate that many changes can be made in the specific aspects which are disclosed and still obtain a like or similar result without departing from the scope of the instant disclosure. It is understood that the examples are given by way of illustration and are not intended to limit the specification of the claims to follow in any manner.

### Example 1

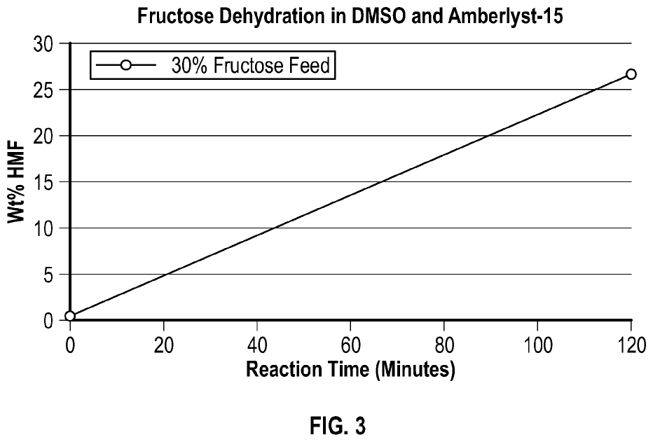

The ability to conduct fructose dehydration using an acid catalyst of the type disclosed herein was investigated. Fructose dehydration in dimethyl sulfoxide with the AMBERLYST catalyst was carried out. Specifically, the conditions were 30 weight percent (wt. %) fructose, 0.2 w/w AMBERLYST-15, 70 wt. % DMSO, and 110° C. in a closed atmosphere. The results are depicted in FIG. 3 which plots the weight percent of 5-hydroxymethylfurfural produced from the dehydration of fructose as a function of time.

### Example 2

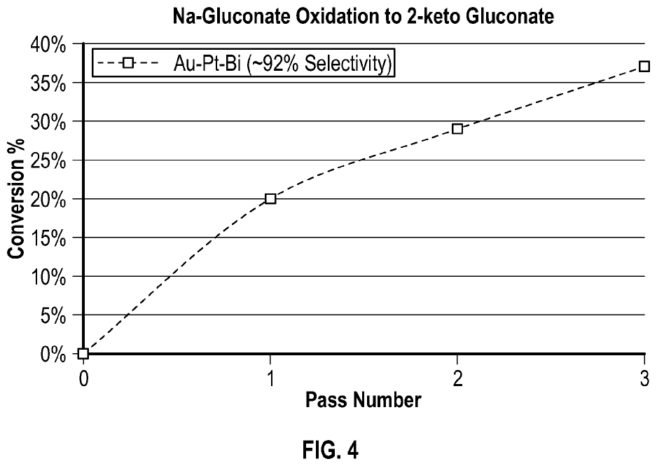

The conversion of gluconic acid to 2-keto gluconic acid using a TMC of the type disclosed herein was carried out. Specifically, gluconic acid and oxygenin the presence of a supported Au/Pt/Bi catalyst were reacted at a temperature of 110° C. by flowing the reactants over the catalyst bed at a rate of 21 ml/min to give a residence time of approximately 7 minutes. The results are presented as a graph of the conversion percentage as a function of pass over the catalyst bed, FIG. 4. The selectivity for this reaction was determined to be 92% which was similar to that of selectivity observed for the oxidation of glucose to gluconic acid carried out using the same catalyst.

### ADDITIONAL DISCLOSURE

The following are non-limiting, specific aspects in accordance with the present disclosure:

A first aspect which is a method of preparing succinic acid; the method comprising contacting glucose with an oxidizing catalyst under conditions to produce a first oxidized product; converting the first oxidized product to an aldehyde; and contacting the aldehyde with an acid catalyst under conditions suitable for the formation of succinic acid.

A second aspect which is the method of the first aspect wherein oxidizing catalyst comprises a transition metal catalyst, an enzymatic catalyst or a combination thereof.

A third aspect which is the method of any of the first through second aspects wherein the enzymatic catalyst comprises an alcohol oxidase.

A fourth aspect which is the method of any of the first through third aspects wherein the alcohol oxidase comprises a mutated galactose oxidase.

A fifth aspect which is the method of any of the first through fourth aspects wherein the transition metal catalyst comprises (i) platinum, bismuth, gold, or a combination thereof and (ii) a support material.

A sixth aspect which is the method of any of the first through fifth aspects wherein the first oxidized product comprises a 2-keto intermediate.

A seventh aspect which is the method of the sixth aspect wherein the 2-keto-intermediate comprises 2-keto gluconic acid.

An eighth aspect which is the method of any of the first through seventh aspects further comprising decarboxylation of the first oxidized product to form a reactant, dehydration of the reactant to form the aldehyde and oxidation of the aldehyde in the presence of an acid catalyst and an oxide to form succinic acid.

A ninth aspect which is the method of any of the first through eighth aspects wherein dehydration is carried out in the presence of an acid catalyst.

A tenth aspect which is the method of any of the first through ninth aspects wherein the decarboxylation is carried out in the presence of a catalyst comprising copper and a polymer.

An eleventh aspect which is the method of any of the first through tenth aspects wherein the 2-keto intermediate is formed with a selectivity of equal to or greater than about 80%.

A twelfth aspect which is the method of any of the first through eleventh aspects wherein the reactant is D-ribulose and is formed at a selectivity of from about 35% to about 70%.

A thirteenth aspect which is the method of any of the first through twelfth aspects wherein the aldehyde is furfural and is formed at a selectivity of equal to or greater than about 50%.

A fourteenth aspect which is the method of any of the first through thirteenth aspects wherein the succinic acid is formed at a selectivity of equal to or greater than about 50%.

A fifteenth aspect which is the method of any of the first through fourteenth aspects wherein the succinic acid has a purity of equal to or greater than about 80%.

A sixteenth aspect which is the method of any of the first through fifteenth aspects wherein the oxidant is hydrogen peroxide.

A seventeenth aspect which is the method of any of the first through sixteenth aspects carried out in a single pot.

An eighteenth aspect which is a solvent produced using the succinic acid of claim 1.

A nineteenth aspect which is the solvent of the eighteenth aspect wherein the solvent comprises dimethyl succinate, diethylsuccinoylsuccinate, succinimide, pyrrolidine, tetrahydrofuran, succinonitrile, gamma-butyrolactone or a combination thereof.

A twentieth aspect which is polybutylene succinate produced from the succinic acid of any of the first through seventeenth aspects.

While aspects of the disclosure have been shown and described, modifications thereof can be made without departing from the spirit and teachings of the presently disclosed subject matter. The aspects and examples described herein are exemplary only, and are not intended to be limiting. Many variations and modifications of the subject matter disclosed herein are possible and are within the scope of the present disclosure.

At least one aspect is disclosed and variations, combinations, and/or modifications of the aspect(s) and/or features of the aspect(s) made by a person having ordinary skill in the art are within the scope of the disclosure. Alternative aspects that result from combining, integrating, and/or omitting features of the aspect(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, 5, 6, . . . greater than 0.10 includes 0.11, 0.12, 0.13, 0.14, 0.15, . . . ). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent . . . 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the detailed description of the presently disclosed subject matter.

1. A method of preparing succinic acid, the method comprising:
contacting glucose with an oxidizing catalyst under conditions to produce a first oxidized product;
converting the first oxidized product to an aldehyde; and
contacting the aldehyde with an acid catalyst under conditions suitable for the formation of succinic acid.

2. The method of claim 1, wherein oxidizing catalyst comprises a transition metal catalyst, an enzymatic catalyst or a combination thereof.

3. The method of claim 2, wherein the enzymatic catalyst comprises an alcohol oxidase.

4. The method of claim 2, wherein the alcohol oxidase comprises a mutated galactose oxidase.

5. The method of claim 2, wherein the transition metal catalyst comprises (i) platinum, bismuth, gold, or a combination thereof and (ii) a support material.

6. The method of claim 1, wherein the first oxidized product comprises a 2-keto intermediate.

7. The method of claim 6, wherein the 2-keto-intermediate comprises 2-keto gluconic acid.

8. The method of claim 1, further comprising decarboxylation of the first oxidized product to form a reactant, dehydration of the reactant to form the aldehyde and oxidation of the aldehyde in the presence of an acid catalyst and an oxide to form succinic acid.

9. The method of claim 1, wherein dehydration is carried out in the presence of an acid catalyst.

10. The method of claim 1, wherein the decarboxylation is carried out in the presence of a catalyst comprising copper and a polymer.

11. The method of claim 1, wherein the 2-keto intermediate is formed with a selectivity of equal to or greater than about 80%.

12. The method of claim 1, wherein the reactant is D-ribulose and is formed at a selectivity of from about 35% to about 70%.

13. The method of claim 1, wherein the aldehyde is furfural and is formed at a selectivity of equal to or greater than about 50%.

14. The method of claim 1, wherein the succinic acid is formed at a selectivity of equal to or greater than about 50%.

15. The method of claim 1, wherein the succinic acid has a purity of equal to or greater than about 80%.

16. The method of claim 1, wherein the oxidant is hydrogen peroxide.

17. The method of claim 1, carried out in a single pot.

18. A solvent produced further comprising the succinic acid of claim 1.

19. The solvent of claim 18, wherein the solvent comprises dimethyl succinate, diethylsuccinoylsuccinate, succinimide, pyrrolidine, tetrahydrofuran, succinonitrile, gamma-butyrolactone or a combination thereof.

20. Polybutylene succinate produced from the succinic acid of claim 1.
